(19) United States
(12) Patent Application Publication
(10) Pub. No.: US 2003/0091986 A1
(43) Pub. Date: May 15, 2003

(54) Identification of expressed genes using phage display

(75) Inventors: Maria G. Pallavicini, Livermore, CA (US); Brian P. Mullaney, Park City, UT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Office of Technology Transfer, Oakland, CA (US)

(21) Appl. No.: 10/014,318

(22) Filed: Nov. 9, 2001

(51) Int. Cl. C12Q 1/70; C12Q 1/68; G01N 33/53

(52) U.S. Cl. 435/5; 435/7.1; 435/6

(57) ABSTRACT

The n provides method of mapping polypeptide-encoding regions of genes. In particular, the invention provides methods of identifying, isolating and mapping a genomic exon sequence at the protein level using epitope phage display libraries. The invention also provides epitope- and antibody-phage display libraries and a novel phage expression vector.

### STATEMENT AS TO RIGHTS TO INVENTIONS MADE UNDER FEDERALLY SPONSORED RESEARCH AND DEVELOPMENT

This invention was made with Government support under Grant No. HL52930, awarded by the National Institutes of Health. The Government has certain rights in this invention.

### BACKGROUND OF THE INVENTION

Validation of candidate gene targets identified by genome sequence analysis frequently requires protein-based strategies. In particular, functional characterization of genes identified by human genome sequencing often requires analysis of protein-protein interactions. Phage display libraries facilitate investigation of the molecular basis of protein-protein interactions (see, e.g., Mullaney, et al., Exper.l Hematol. in press, 2001). For example, phage display peptide libraries (e.g., Scott et al., Science 249, 386-390, 1990) have been used to characterize antibody-epitope interactions (see, e.g., Cortese et al., Curr Opin Biotechnol. 7:616-621, 1996; Burton, D. R., Immunotechnology 1:87-94, 1995; Fack et al., J Immunol Methods 206:43-52, 1997) and phage display cDNA libraries have been used to define a variety of protein-protein interactions (see, e.g., Santi et al., J Mol Biol. 296:497-508, 2000; Pereboeva, et al., J Med Virol. 60:144-151, 2000; Hufton et al., J Immunol Methods 231:39-51, 1999; Cochrane et al., J Mol Biol. 297:89-97, 2000; and Zozulya et al., Nat Biotechnol. 17:1193-1198, 1999).

Identification of coding regions is a key step in linking genome sequence with expressed proteins. Computational analysis of DNA sequence has been used extensively to predict coding regions. Protein-based methodologies that enrich coding (exon) sequences from non-coding sequences can complement computational approaches because such methods can facilitate linkage of genotype with protein phenotype. Genome-protein linkage is particularly relevant for diseases, such as cancer or various inherited diseases, where genomic alterations (i.e., amplification, deletion, translocation, etc.) are prevalent, yet the spectrum of expressed genes encoded and expressed by these altered regions is often unknown.

Identification of disease-related genes is a multi-step, labor intensive process. Typically, disease-related genomic intervals are identified and mapped using linkage analyses for inherited disorders or genome wide survey techniques, such as chromosome banding, comparative genomic hybridization (Kallioniemi (1992) Science 258: 818-21) or loss of heterozygosity (Cher (1994) Genes, Chromosomes & Cancer 11:153-162). Mapping of a disease-related genomic region typically begins with the identification of a chromosomal region ranging from one to ten centimorgans containing as many as 100 to 1000 genes. Even with sequence information available for the chromosomal region, these gene identification and mapping processes are laborious and time-consuming. Furthermore, most nucleic acid sequences and genes in a chromosomal region suspected of being associated with a disease are not involved in the genetically-linked disease. Many may not even be expressed in the affected tissue. An approach to rapidly link a gene sequence in a chromosomal region suspected of being associated with a disease with expressed proteins in the affected tissue would greatly facilitate identification of disease-associated genes. For example, this concept is useful in cancer genetics where multiple regions of recurrent genomic alteration are identified.

Phage display has been used to display small genomes, such as Hepatitis C virus (e.g., Santi, supra, and Pereboeva, supra) or prokaryotic artificial chromosomes (e.g., Fehrsen et al., Immunotechnology 4:175-184, 1999; Jacobsson et al., Biotechniques 18:878-885, 1995; and Jacobsson et al., Biotechniques 20:1070-1076, 1078, 1080-1071, 1996). However, the technique has not been applied to mapping eukaryotic, e.g., mammalian or human, genomic fragments to identify peptides encoded by regions of the genome that may contain candidate genes that have not been confirmed or to identify expressed genes in genomes or genomic regions that have not yet been characterized or sequenced.

The current invention provides method of mapping polypeptide-encoding regions of genomic nucleic acid. In particular, the invention provides methods of identifying, isolating and mapping a genomic exon sequence at the protein level using epitope phage display libraries. The invention also provides epitope- and antibody-phage display libraries and a novel phage expression vector.

### BRIEF SUMMARY OF THE INVENTION

The invention provides a method of identifying an exon in a genomic fragment, e.g., a eukaryotic genomic fragment. The method comprises expressing a population of subsequences of the genomic fragment in a phage display library. The population comprises both protein-encoding subsequences and noncoding subsequences. The library is screened with a binding partner to identify an expressed subsequence that specifically binds to the binding partner; and the expressed subsequence is mapped to its physical location in the genomic fragment. The binding partner is typically an antibody, an enzyme, or a receptor and can be expressed by a phage display library. In some embodiments, in which the binding partner is an antibody, the antibody is a single chain antibody, e.g., a single chain Fv antibody (scFv).

The expressed subsequences are typically at least about 100, often 150, base pairs in length and no longer than about 300 base pairs in length. These sizes are often the sizes of exons. The genomic fragment is often from a mammalian genome and in some embodiments, the identified exon is abnormally expressed in a cell of an individual with a disease, such as cancer.

The population of subsequences in the phage display library also comprises noncoding subsequences, i.e., sequences that do not encode a polypeptide in vivo. For example, the noncoding subsequence can be from an intron, or can comprise reptetitive DNA sequences such as Alu or Kpn repeat sequences.

The invention also provides a phage display library comprising phage that express a population of subsequences of a eukaryotic genomic fragment, often a fragment from a mammalian genome. The population comprises protein coding subsequences and noncoding subsequences. In some embodiments, the eukaryotic genomic fragment is from a mammalian genome.

The library can be constructed using a vector such as a pBPM-1 vector. Often, the size of the inserts is from about 100 base pairs to about 300 base pairs in length.

The invention also provides a phage expression vector comprising a polylinker region, an out-of-frame pIII gene, and at least one non-pallindromic rare cutting restriction enzyme site, e.g., an SfiI site, located in the polylinker site, wherein the non-pallindromic rare cutting restriction enzyme site is not located outside the polylinker region, and a selection tag encoding sequence. The selection tag can be an epitope tag selected from the group consisting of a polyhistidine tag or a myc tag or can be an antibiotic resistance polypeptide. An example of the vector is the pBPM-1 vector.

### BRIEF DESCRIPTION OF THE DRAWINGS

FIG. 1. Theoretical considerations for genomic epitope display of 5q31. All open reading frames from the 50 kb P1H11 were calculated and compared to exon size of 5q31 genes. The probability of a stop codon within a given fragment size is plotted.

FIG. 2. Size distribution of PCR inserts from unselected H11 epitope phage library. Insert sequence of individual random clones was amplified using PCR primers that flank the insert cloning site and analyzed on a 2.0% agarose gel.

FIG. 3. Specificity of mimotope clones for IL-4 by displacement ELISA. The anti-IL-4 antibody, C19, was preincubated with or without increasing concentrations (0-20 mg/ml) of specific blocking peptide SC-1260 prior to ELISA with phage epitope (H11_{—}207) and mimotope (H11_{—}201) clones. (H11_{—}201 without peptide, circle; H11_{—}201 with peptide, square: H11_{—}207 with peptide, diamond). Data are representative of two experiments.

Definitions

A “noncoding subsequence” refers to a region of a genomic fragment that does not encode a protein sequence in vivo. Such sequence include both transcribed, e.g., introns, and nontranscribed sequences. A “repetitive sequence” or “repetitive element” refers to regions of the genome that are repeated, e.g., LINES, SINES, variable number tandem repeat sequences (VNTRs) and the like.

A “binding partner” refers to a molecule that participates in a specific binding interaction with a peptide that is displayed on a library. The binding partner can also be referred to as a “second binding pair member” or “cognate binding partner”. Peptide/binding partner pairs include antibodies/antigens, receptor/ligands, and interacting protein domains such as leucine zippers and the like. A binding partner as used herein can be a binding domain, i.e., a subsequence of a protein that binds specifically to a display peptide. A binding partner is often a protein, but can be any molecule that binds specifically to a displayed peptide, e.g., a nucleic acid, a polysaccharide, or the like.” A polypeptide binding partner can be an antibody, an antigen-binding fragment of an antibody, an enzyme, an intra- or extra-cellular receptor, a protein binding lipid, a cis-acting transcriptional or translational regulatory region of a gene or transcript, and the like.

The term “mapping an expressed subsequence” refers to identifying the physical location of a nucleic acid sequence on the genomic fragment. Mapping the expressed subsequence typically comprises sequencing the nucleic acid encoding the expressed subsequence and determing its location on the genomic fragment used to prepare a phage diplay library of the invention. The physical location of the expressed sequence on a chromosome can also be determined, for example, by determining the physical relationship of of the sequence to a genetic linkage map or other relevant chromosomal landmarks, such as banding patterns, chromosomal rearrangements, or the location of known genes.

“Enriching” refers to at least one, preferably two or more, rounds of selection to increase the proportion of exon-expressing subsequences in the peptide display library.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments can be synthesized de novo, often using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

As used herein, the term “single-chain antibody” refers to a polypeptide comprising a V_{H} domain and a V_{L} domain in polypeptide linkage, generally linked via a spacer peptide (e.g., [Gly-Gly-Gly-Gly-Ser]ₓ), and which may comprise additional amino acid sequences at the amino- and/or carboxy-termini. For example, a single-chain antibody may comprise a tether segment for linking to the encoding polynucleotide. As an example, a scFv is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp. 361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope). Techniques for the production of single chain antibodies (U.S. Pat. No. 4,946,778) can be adapted to produce antibodies for use in this invention.

The term “condition” refers to any physiologic state that is not optimally normal or healthy, including, e.g., a stress, an injury, infection, disease, pathology, drug side effect, contamination (as e.g., a pollutant), poisoning, irritation, or predisposition (e.g., as in a genetic predisposition) thereof.

“Domain” refers to a unit of a protein or protein complex, comprising a polypeptide subsequence, a complete polypeptide sequence, or a plurality of polypeptide sequences where that unit has a defined function. The function is understood to be broadly defined and can be binding to a binding partner, catalytic activity or can have a stabilizing effect on the structure of the protein.

“Link” or “join” refers to any method of functionally connecting peptides, including, without limitation, recombinant fusion, covalent bonding, disulfide bonding, ionic bonding, hydrogen bonding, and electrostatic bonding. In the systems of the invention, a binding pair member is typically fused, using recombinant DNA techniques, at its N-terminus or C-terminus to a reporter molecule or to an activator or inhibitor of the reporter molecule. The reporter molecule can be a complete polypeptide, or a fragment or subsequence thereof. For example, a binding pair member can be linked to a complementing fragment of a reporter molecule. The binding pair member can either directly adjoin the fragment to which it is linked or can be indirectly linked, e.g., via a linker sequence.

“Fused” refers to linkage by covalent bonding.

A “fusion protein” refers to a protein comprising at least one polypeptide or peptide domain that is linked or joined to a second domain. The second domain can be a polypeptide, peptide, polysaccharide, or the like. If the polypeptides are recombinant, the “fusion protein” can be translated from a common message.

As used herein, “isolate,” when referring to a molecule or composition, such as, for example, a polypeptide or nucleic acid or phage, means that the molecule or composition is separated from at least one other compound, such as a protein, other nucleic acids (e.g., RNAs), or other contaminants with which it is associated in vivo or in its naturally occurring state. Thus, a nucleic acid or phage is considered isolated when it has been isolated from any other component with which it is naturally associated, e.g., cell membrane, as in a cell extract. An isolated composition can, however, also be substantially pure. An isolated composition can be in a homogeneous state and can be in a dry or an aqueous solution. Purity and homogeneity can be determined, for example, using analytical chemistry techniques such as polyacrylamide gel electrophoresis (SDS-PAGE) or high performance liquid chromatography (HPLC).

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties, for the purposes desired, as the reference nucleic acid. The term also includes nucleic acids which are metabolized in a manner similar to naturally occurring nucleotides or at rates that are improved thereover for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see, e.g., Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev. 6:153-156). The term nucleic acid is used interchangeably with gene, cDNA, mRNA, oligonucleotide primer, probe and amplification product.

A “phage display library” refers to a “library” of bacteriophages on whose surface is expressed exogenous peptides or proteins. The foreign peptides or polypeptides are displayed on the phage capsid outer surface as recombinant fusion proteins incorporated as part of a phage coat protein. This is accomplished by inserting an exogenous nucleic acid sequence into the coding sequence of a phage coat protein. If the foreign sequence is “in phase” the protein it encodes will be expressed as part of the coat protein. Thus, libraries of nucleic acid sequences, such as a genomic library from a specific cell or chromosome, can be so inserted into phages to create “phage libraries.” As peptides and proteins representative of those encoded for by the nucleic acid library are displayed by the phage, an “epitope-display library” or “antibody-display library” is generated. While a variety of bacteriophages are used in such library constructions, typically, filamentous phage are used (Dunn (1996) Curr. Opin. Biotechnol. 7:547-553). See, e.g., description of phage display libraries, below.

A “phage expression vector” or “phagemid” refers to any phage-based recombinant expression system for the purpose of expressing a nucleic acid sequence in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

A “peptide encoded by one or more DNA sequences which are not translated in vivo” refers to a peptide or polypeptide which is not normally produced in vivo, i.e., the term refers to translation products of normally non-transcribed nucleic acid, which nucleic acid, when cloned, as in an epitope library or a vector, can generate an mRNA and protein.

### DETAILED DESCRIPTION OF THE INVENTION

Introduction

This invention relates to a novel approach to discover, isolate and map new genes at the protein level using phage display libraries. The methods of the invention use phage display libraries to rapidly associate genomic nucleic acid sequences with expressed mRNAs and corresponding polypeptides in a target cell or tissue. This “peptide trapping” approach provides a rapid means to associate protein expression with defined genomic intervals, i.e., it is a quick and efficient way to map and identify exon-coding genomic sequences. Thus, the methods and libraries of the invention are valuable for linking phenotype with genotype, thereby providing a new means for identifying genes, for example, genes expressed in a particular condition or disease state, or expressed genes from an uncharacterized region of a genome.

Genes encoding proteins whose expression is associated with a particular phenotype, i.e., a cell or tissue type, a disease or a condition, a developmental state, a stage in the cell cycle, can be rapidly identified and mapped with the methods of the invention. Similarly, genes encoding proteins responsive to a stimulus, such as a chemical, pharmacologic, environmental or metabolic stimulus can be so mapped. In genetically altered tissues with chromosomal rearrangements, mutations or amplifications, epitope-expressing sequences effected by the genetic alteration can also be rapidly identified and mapped.

The methods of the invention involve identifying a phage in a peptide-expressing phage display library that expresses a protein sequence of interest. In some embodiments, the phage display library expresses genomic DNA from a previously mapped chromosomal segment. This allows rapid identification of the physical region of the chromosome encoding the polypeptide reacting with the binding partner. This chromosomal preselection is possible if it there is a high likelihood that the epitope of interest is expressed by a particular subregion. For example, it is known that a subsection of chromosome 5, 5q31, encodes a variety of hematopoietic and immune cell antigens. If the objective is to map genes encoding for polypeptides expressed on hematopoietic cells, a library expressing this defined subset of chromosome 5, known to encode hematopoietic antigens, is selected.

In many instances, however, a particular chromosomal region cannot be preselected. In these cases, libraries encompassing an entire genome or regions of a genome, e.g., individual chromosomes or chromosomal regions, can be initially screened.

This invention provides for novel epitope phage display libraries, antibody phage display libraries, phage expression vectors, and methods for the discovery, isolation, sequencing and mapping of genomic exon sequences. The invention can be practiced in conjunction with any method or protocol known in the art, which are well described in the scientific and patent literature. Therefore, only a few general techniques are described herein prior to discussing specific methodologies and examples relative to the novel reagents and methods of the invention.

The techniques for constructing and analyzing phage display libraries uses recombinant technology well known to those of skill in the art. General techniques, e.g., manipulation of nucleic encoding libraries, epitopes, antibodies, and vectors of interest, generating libraries, subcloning into expression vectors, labeling probes, sequencing DNA, DNA hybridization are described in the scientific and patent literature, see e.g., Sambrook and Russell, eds., Molecular Cloning: a Laboratory Manual (3rd), Vols. 1-3, Cold Spring Harbor Laboratory Press, (2001) (“Sambrook”); Current Protocols in Molecular Biology, Ausubel, ed. John Wiley & Sons, Inc., New York (1997-2001) (“Ausubel”); and, Laboratory Techniques in Biochemistry and Molecular Biology: Hybridization with Nucleic Acid Probes, Part I. Theory and Nucleic Acid Preparation, Tijssen, ed. Elsevier, N.Y. (1993) (“Tijssen”). Sequencing methods typically use dideoxy sequencing, however, other methodologies are available and well known to those of skill in the art.

Nucleic acids and proteins are detected and quantified in accordance with the teachings and methods of the invention by any means known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, Southern analysis, Northern analysis, Dot-blot analysis, gel electrophoresis (e.g., SDS-PAGE), RT-PCR, quantitative PCR, other nucleic acid or target or signal amplification methods, radiolabeling, scintillation counting, and affinity chromatography.

Phage Display Library

Construction of Phage Display Libraries

Construction of phage display libraries exploits the bacteriophage's ability to display peptides and proteins on their surfaces, i.e., on their capsids. Often, filamentous phage such as M13 or fl are used. Filamentous phage contain single-stranded DNA surrounded by multiple copies of genes encoding major and minor coat proteins, e.g., pIII. Coat proteins are displayed on the capsid's outer surface. DNA sequences inserted in-frame with capsid protein genes are co-transcribed to generate fusion proteins or protein fragments displayed on the phage surface. Peptide phage libraries thus can display peptides representative of the diversity of the inserted genomic sequences. Significantly, these epitopes can be displayed in “natural” folded conformations. The peptides expressed on phage display libraries can then bind target molecules, i.e., they can specifically interact with binding partner molecules such as antibodies (Petersen (1995) Mol. Gen. Genet. 249:425-31), cell surface receptors (Kay (1993) Gene 128:59-65), and extracellular and intracellular proteins (Gram (1993) J. Immunol. Methods 161:169-76).

The concept of using filamentous phages, such as M13 or fd, for displaying peptides on phage capsid surfaces was first introduced by Smith (1985) Science 228:1315-1317. Peptides have been displayed on phage surfaces to identify many potential ligands (see, e.g., Cwirla (1990) Proc. Natl. Acad. Sci. USA 87:6378-6382). There are numerous systems and methods for generating phage display libraries described in the scientific and patent literature, see, e.g., Sambrook and Russell, Molecule Cloning: A Laboratory Manual, 3rd edition, Cold Spring Harbor Laboratory Press, Chapter 18, 2001; “Phage Display of Peptides and Proteins: A Laboratory Manual, Academic Press, San Diego, 1996; Crameri (1994) Eur. J. Biochem. 226:53-58; de Kruif (1995) Proc. Natl. Acad. Sci. USA 92:3938-42; McGregor (1996) Mol. Biotechnol. 6:155-162; Jacobsson (1996) Biotechniques 20:1070-1076; Jespers (1996) Gene 173:179-181; Jacobsson (1997) Microbiol Res. 152:121-128; Fack (1997) J. Immunol. Methods 206:43-52; Rossenu (1997) J. Protein Chem. 16:499-503; Katz (1997) Annu. Rev. Biophys. Biomol. Struct. 26:27-45; Rader (1997) Curr. Opin. Biotechnol. 8:503-508; Griffiths (1998) Curr. Opin. Biotechnol. 9:102-108.

Typically, exogenous nucleic acid to be displayed are inserted into a coat protein gene, e.g. gene III or gene VIII of the phage. The resultant fusion proteins are displayed on the surface of the capsid. Protein VIII is present in approximately 2700 copies per phage, compared to 3 to 5 copies for protein III (Jacobsson (1996), supra). Multivalent expression vectors, such as phagemids, can be used for manipulation of exogenous genomic or antibody encoding inserts and production of phage particles in bacteria (see, e.g., Felici (1991) J. Mol. Biol. 222:301-310).

Phagemid vectors are often employed for constructing the phage library. These vectors include the origin of DNA replication from the genome of a single-stranded filamentous bacteriophage, e.g., M13 or fl. A phagemid can be used in the same way as an orthodox plasmid vector, but can also be used to produce filamentous bacteriophage particle that contain single-stranded copies of cloned segments of DNA.

Other phage can also be used. For example, T7 vectors can be employed in which the displayed product on the mature phage particle is released by cell lysis.

Another useful methodology is selectively infective phage (SIP) technology. which provides for the in vivo selection of interacting protein-ligand pairs. A “selectively infective phage” consists of two independent components. A recombinant filamentous phage particle is made non-infective by replacing its N-terminal domains of gene 3 protein (g3p) with a ligand-binding protein. For example, the genomic nucleic acid to be mapped can be inserted such that it will be expressed as this ligand-binding protein. The second component is an “adapter” molecule in which the ligand is linked to those N-terminal domains of g3p which are missing from the phage particle. Infectivity is restored when the displayed protein (e.g., a “binding site”) binds to the epitope ligand. This interaction attaches the missing N-terminal domains of g3p to the epitope phage display particle. Phage propagation becomes strictly dependent on the protein-ligand interaction. See, e.g., Spada (1997) J. Biol. Chem. 378:445-456; Pedrazzi (1997) FEBS Lett. 415:289-293; Hennecke (1998) Protein Eng. 11:405-410.

Construction of Non-Phage Display Libraries

In addition to phage epitope display libraries, analogous epitope display libraries can also be used. For example, the methods of the invention can also use yeast surface displayed epitope libraries (see, e.g., Boder (1997) “Yeast surface display for screening combinatorial polypeptide libraries,” Nat. Biotechnol. 15:553-557), which can be constructed using such vectors as the pYD1 yeast expression vector. Other potential display systems include mammalian display vectors and E. coli libraries.

Sources of Genomic DNA: Microsatellites and Clones

The invention provide methods using phage display libraries which contain subsequences of a genomic fragment. The genomic fragment is typically from a mapped region, i.e., a regions for which the physical location of the fragment in the genome, for example the location in a chromosome or chromosomal regions is known. Use of mapped genomic DNA to construct the phage display libraries allows for rapid linking of a protein sequence coding region to a physical location on a chromosome. Sources of mapped genomic DNA include microsatellites (see, e.g., Dib (1996) Nature 380:152-154), YACs, BACs, P1 or cosmid genomic libraries. BACs, bacterial artificial chromosomes, are vectors that can contain 120+ Kb inserts. BACs are based on the E. coli F factor plasmid system and simple to manipulate and purify in microgram quantities. Yeast artificial chromosomes, or YACS, contain inserts ranging in size from 80 to 700 kb, see, e.g., Tucker (1997) Gene 199:25-30; Adam (1997) Plant J. 11:1349-1358. P1 is a bacteriophage that infects E. coli that can contain 75-100 Kb DNA inserts (Mejia (1997) Genome Res 7:179-186; Ioannou (1994) Nat Genet 6:84-89), and are screened in much the same way as lambda libraries.

Publicly available electronic databases are rapid sources of microsatellites, chromosomal maps, genomic sequences, and the like, see, e.g., Généthon Microsatellite Maps; or GenLink; or GenBank Sequence Database.

Construction of Genomic Libraries

The invention provides an epitope phage display library where the phages in the library express one or more protein epitopes encoded by one or more fragments of a genomic exon sequence. The invention also provides methods for identifying, isolating and mapping a genomic exon sequence at the protein level involving screening epitope phage display libraries with a binding partner, such as a receptor or an antibody. The epitope phage display libraries can be constructed by inserting fragmented genomic DNA in the coat protein coding region of the phage, as discussed above. The genomic nucleic acid can be representative of an entire genome, a particular chromosome, or from a defined chromosomal segment (as used in Example 1). The invention also provides a method of mapping a genomic exon sequence whose expression is increased or activated, or decreased or inactivated, by a stimulus to a cell using a phage display library expressing cDNA encoded epitopes.

This invention provides a phage display strategy to identify coding exon sequences from regions of a genome. For example, epitope phage display libraries from specific regions of the human genome can be enriched for coding exon sequences that bind to target proteins such as antibodies. The methods of the invention maximize the likelihood of exon display, library diversity, and minimize introns and stop codons. Peptides generated from genomic fragments will encode primarily linear, small exon-specific epitopes. Longer exons may encode discontinuous conformational epitopes.

Other considerations involve the number of introns expected to be present in the eukaryotic sequence relative to the number of exons. For example, in a species that has a relatively low number of introns relative to exons, the size of the subsequences inserted into the phage display vector can be larger. However, the size of the fragment also has ramifications for the size of the library as the library must contain enough members to represent all or the vast majority of the genomic fragment to be analyzed using the methods of the invention.

Methods for making genomic libraries are also well known, see e.g., Sambrook, Ausubel, Tijssen. In one exemplary means to make a genomic library, DNA, for example corresponding to the gene fragment to be analyzed using the methods of the invention, is extracted, purified and fragmented into subsequences fragments. Fragmented genomic nucleic acid of appropriate size is produced by known methods, such as nebulization, mechanical shearing or enzymatic digestion, to yield DNA fragments. While the genomic subsequences for cloning into the phage library can be any size, e.g., of about 45 base pairs to 20 kb, the fragments inserted in phage are are often at least about 75, 100, 125, 150, 175, 200, or 250 base pairs in length. In a preferred embodiment, the fragments are at least about 150 base pairs in length. The upper limit of fragments inserted into the phage can vary, depending on the length of the exons that are suspected of being contained in the genomic fragment that is being mapped for exons. Typically the fragment is no longer than about 5,000 base pairs in length, e.g., 3000, 2000, 1500, 1000, 500, 400, 350, or about 300 base pairs. In preferred embodiments, the fragments are about 150 to 300 bases in size.

The rationale for this size restriction is based on the intron-exon pattern of gene structure. For example, in silico sequence analyses of the 5q31 Interleukin gene region indicates that the majority of the exons within this region range between 100-300 bp. Variables related to genomic sequence, such as size of the target region (kilobase, megabase, etc.), gene location within six reading frames, stop codon frequency and in-frame sequences are important considerations in developing phage display-based coding exon identification. In addition, proper cloning orientation is required for successful phage display. An insert sequence must be in-frame relative to the leader sequence and continue in-frame into the phage display framework sequence (e.g., Cabilly, Mol. Biotechnol. 12:143-148, 1999). A stop codon within the insert sequence will cause a premature truncation of the peptide and prevent surface display.

For a peptide to be successfully displayed by the phage, an insert sequence must be in-frame in relationship to the leader sequence and continue in-frame into the display framework, e.g., the pIII sequence. Any stop codon (TGA, TAA, TAG) within the insert sequence will cause a premature truncation of the peptide and prevent surface display. Intron DNA contains stop codon sequences at approximately a frequency similar to random DNA. The probability of a stop codon occurring in random sequence length is calculated as 4.7% (3 stop codons per 64 total codons) per amino acid or DNA triplet. Approximately 90% of random sequences will terminate by about 50 amino acids, i.e., after about 150 base pairs (bp). Thus, using a 150 bp lower limit for library insert size will minimize expression of the majority of intron DNA sequences.

In contrast, selection of an upper limit for library inserts is based on exon size. For example, the average exon size for known genes on the chromosomal fragment 5q31 is approximately 100 to 150 bp. Gene exon fragments also may display some flanking introns. Thus, the upper limit may be considered as 300 bp (150 bp exon plus 150 bp of random sequence). Selecting a size range of fragments within the limits of about 150 bp and about 300 bp therefore easily allows full coverage of the entire 5q31 sequence, within the limitations of library construction.

Once the genomic DNA being analyzed has been fragment, the genomic nucleic acid fragments of desired size are then separated, e.g., by gradient centrifugation, or gel electrophoresis, from undesired sizes. The sizes of the fragments included in the desired population range can vary. For example, a desired population of from about 150 to about 300 base pairs can contain fragments of other sizes that are smaller than 150 or larger than 300 base pairs. The fragments are inserted in bacteriophage or other vectors. The vectors and phage can be packaged in vitro or in vivo. Recombinant phage can be analyzed by plaque hybridization described, e.g., in Benton (1977) Science 196:180; Chen (1997) Methods Mol Biol 62:199-206. Colony hybridization can be carried out as generally described in the scientific literature, e.g., as in Grunstein (1975) Proc. Natl. Acad. Sci. USA 72:3961-3965; Yoshioka (1997) J. Immunol Methods 201:145-155; Palkova (1996) Biotechniques 21:982.

Amplification of Nucleic Acids

Nucleic acids can also be generated for subcloning into a phage display vector using any amplification methodology known in the art using a variety of hybridization techniques and conditions. Amplification can be used for, e.g., the construction of hybridization probes or clones, identification, sequencing, quantification, and the like. Amplification primer pairs can be used to screen for the presence of antibody- or epitope-encoding nucleic acid sequences in a sample. Suitable amplification methods include, but are not limited to: polymerase chain reaction, PCR (PCR Protocols, A Guide to Methods and Applications, ed. Innis, Academic Press, N.Y. (1990) and PCR STRATEGIES (1995), ed. Innis, Academic Press, Inc., N.Y. (Innis)), ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and, self-sustained sequence replication (Guatelli (1990) Proc. Natl. Acad. Sci. USA, 87:1874); Q Beta replicase amplification and other RNA polymerase mediated techniques (e.g., NASBA, Cangene, Mississauga, Ontario); see Berger (1987) Methods Enzymol. 152:307-316, Sambrook, and Ausubel, as well as Mullis (1987) U.S. Pat. Nos. 4,683,195 and 4,683,202; Arnheim (1990) C&EN 36-47; Lomell J. Clin. Chem., 35:1826 (1989); Van Brunt, Biotechnology, 8:291-294 (1990); Wu (1989) Gene 4:560; Sooknanan (1995) Biotechnology 13:563-564. Methods for cloning in vitro amplified nucleic acids are described in Wallace, U.S. Pat. No. 5,426,039. Methods of amplifying large nucleic acids are summarized in, e.g., Cheng (1994) Nature 369:684-685.

For example, PCR can be used in a variety of protocols to amplify, identify, quantify, isolate and manipulate nucleic acids. In these protocols, primers and probes for amplification and hybridization are generated that comprise all or any portion of the DNA sequences described herein.

PCR-amplified sequences can also be labeled and used as detectable probes. The labeled amplified DNA or other oligonucleotide or nucleic acid of the invention can be used as probes to further identify and isolate, or identify and quantify, exons or antibody-encoding sequences from any source of nucleic acid, including, RNA, cDNA, genomic DNA, genomic libraries, in situ nucleic acid, and the like.

Binding Partners Reactive with Protein Epitopes

In the methods of the invention, a second component in identifying a phage expressing a sequence encoded by an exon involves providing a binding partner specifically reactive with the protein. The binding partner can be any protein of interest, such as an antibody, a receptor or an enzyme. The binding partner can be a library of molecules specifically expressed on a cell or tissue type, or disease state, or the like.

If the binding partner is an antibody, it can be a monoclonal, polyclonal or a phage-displayed antibody. The antibodies can be designed to be specifically reactive with a particular set of molecules, cells, or tissues. Antibodies specific for any cell or tissue type, or stage of development or differentiation, or level of activation or inactivation, or the like, can be used. A library of nucleic acids encoding these set of antibodies can be generated. For example, as described in Example 1, antibodies generated against hematopoietic cells which react with phages displaying epitopes encoded by 5q31-located exons are selected. Once the epitope-encoding nucleic acid is isolated from the selected phage, its specific physical location on a chromosome can be rapidly identified.

Other binding partners, such as receptors or enzymes, can also expressed by a phage display library.

The antibody phage-display libraries can also express binding partner polypeptides that are antibody-like molecules, as described, e.g., by Marks (1996) N. Engl. J. Med. 335: 731-733. These antibody phage-display libraries can include DNA sequences that encode the epitope-binding portions of heavy- and light-chain variable regions of immunoglobulin (Ig); see, e.g., Marks (1992) J. Biol. Chem. 267: 16007-10; Griffiths (1993) EMBO J. 12: 725-734. Alternatively, the displayed protein can be a “single-chain” (scFv) Ig fragment (see, e.g., Pistillo (1997) Exp. Clin. Immunogenet. 14:123-130.

Construction of Antibody Libraries

Immunization to generate anti-target cell (e.g., anti-hematopoietic cell) antibodies can be by any means, e.g., injection of cell or membrane extracts, recombinant expression and isolation of target cell translation products, or use of hematopoietic cell naked DNA to directly express antigenic protein in the antibody-generating host (see, e.g., Manickan (1997) Crit. Rev. Immunol. 17:139-154).

The antibody can be single or double-chained, or merely an antigen binding fragment. The antibody can be expressed on the surface of a phage, as in an antibody phage display library, as described above. The antibody binding partner can be a monoclonal antibody or a set of polyclonal antibodies. Methods of producing polyclonal and monoclonal antibodies are known to those of skill in the art and described in the scientific and patent literature, see, e.g., Coligan, Current Protocols in Immunology, Wiley/Greene, NY (1991); Stites (eds.) Basic and Clinical Immunology (7th ed.) Lange Medical Publications, Los Altos, Calif.; Goding, Monoclonal Antibodies: Principals and Practice (2d ed.) Academic Press, New York, N.Y. (1986); Kohler (1975) Nature 256:495; Harlow and Lane, supra. See, Hayden (1997) Curr. Opin. Immunol. 9:201-212, for a review on recombinant antibody engineering techniques. The isolation of a high-affinity stable single-chain antibody, “scFv,” is described, e.g., by Chowdhury (1998) Proc. Natl. Acad. Sci. USA 95:669-674. Such techniques can include selection of antibodies from libraries of recombinant antibodies displayed in phage, or other cells (production of antibody phage display libraries is discussed above, see also, Huse (1989) Science 246:1275 and Ward (1989) Nature 341:544). Recombinant antibodies can be expressed by transient or stable expression vectors in mammalian cells, as in Norderhaug (1997) J. Immunol. Methods 204:77-87.

Alternatively, a high complexity naive library (Marks (1991) J. Mol. Biol. 222:581-597) can be used to select single chain (“scFv”) or double chain antibodies against a cell or tissue type to bypass the requirement for immunization (see, e.g., Aujame (1997) Hum Antibodies 8:155-168). Only a single exon-epitope identified by one antibody displaying phage is required to identify a gene. Thus, epitope trapping will be successful using an antibody phage display library generated from only moderate immune response or a high complexity naive library.

The antibody libraries can be from a number of sources. The some embodiments, the invention provides antibody phage display libraries expressing the equivalent of message from activated B cells, wherein the B cells were activated by immunization with a nucleic acid whose expression is increased or activated, or decreased or inactivated, by a stimulation to the cell. Antibody phage libraries generated using cDNA from Ig gene message from B cells retain the specificity and diversity of the parent antibodies, i.e., the antibodies which would have been generated by the B cells from which the Ig message was harvested. Thus, the antibody repertoire (the specificities of the expressed antibodies) of an antibody phage display library generated using cDNA from message of stimulated B cells reflects the same antibody repertoire of what would be a primary (or secondary, if from a boosted animal) immune response. Such libraries can be used to screen the peptide phage display libraries of the invention that express subsequences of a genomic fragment.

Synthesis of Polypeptide Binding Partners

In the methods of the invention, binding sites are reacted with phage display libraries to screen and isolate exon-encoding phages. The binding partners can be receptors, enzymes, antibodies, and the like. The binding sites can be isolated (from natural sources), synthetic, or recombinantly generated. If the binding sites are peptides, polypeptides or nucleic acids, they can be recombinantly expressed in vitro or in vivo. These peptides and polypeptides can be made and isolated using any method known in the art. Antibodies as binding partners are discussed above.

The binding partners can be synthesized, whole or in part, using chemical methods well known in the art (see e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. (“Banga”)). For example, peptide synthesis can be performed using various solid-phase techniques (see, e.g., Roberge (1995) Science 269:202; Merrifield (1997) Methods Enzymol. 289:3-13) and automated syntheses (e.g., an ABI 431A Peptide Synthesizer, Perkin Elmer).

Synthesized polypeptides or peptides can be isolated and substantially purified by preparative high performance liquid chromatography (HPLC), see, e.g., Creighton, Proteins, Structures and Molecular Principles, W H Freeman and Co, New York N.Y., 1983. The composition of the synthetic protein may be confirmed by amino acid analysis or sequencing (e.g., the Edman degradation procedure; Creighton, supra). Laser desorption mass spectrometry (MALDI-MS) can also be used to evaluate the progress of protein synthesis at all the necessary levels, including automated assembly, cleavage and deprotection chemistries, RP-HPLC analyses and purifications, and structural validation of the final product (Moore (1997) Methods Enzymol. 289:520-542). Electrospray ionization mass spectrometry is useful for verification of peptide synthesis and for the identification of most synthetic by-products (Burdick (1997) Methods Enzymol. 289:499-519).

Amino acid sequences of the binding partner peptides and polypeptides, or any part thereof, can be modified during direct synthesis and/or combined using chemical methods with sequences from other proteins, or any part thereof, to produce variants. Modified proteins can also be produced by manipulation of nucleic acid coding sequence, e.g., with site-directed mutagenesis, or chemical modification of polypeptide to introduce unnatural amino acid side chains (see e.g., Paetzel (1997) J. Biol. Chem. 272:9994-10003, for general methodology). For site-specific incorporation of unnatural amino acids into proteins in vivo, see e.g., Liu (1997) Proc. Natl. Acad. Sci. USA 94:10092-10097; see also Koh (1997) Biochemistry 36:11314-11322; Gallivan (1997) Chem. Biol. 4:739-749.

Cell surface polypeptides can also be isolated from a natural sources, such as a cell line expressing the desired antigens or a patient with a particular disease, condition or genotype, using a variety of techniques well known in the art. Such isolates can be used as immunogens to generate binding partners to be used in the methods of the invention, i.e., to identify, isolate and map genes expressed in a specific cell type, such as hematopoietic cells, as described in Example I. For example, the cells can be solubilized by treatment with papain, by treatment with 3M KCl, or by treatment with detergent. Detergent can then be removed by dialysis, affinity chromatography (e.g., using lectins, or previously tagged cell surface proteins). The molecules can be obtained by isolation from any cell expressing a molecule of interest using standard techniques, e.g., molecules can be separated using SDS/PAGE and electroelution, ion exchange chromatography, size exclusion chromatography, gel permeation chromatography, HPLC, and the like.

Screening Peptides with Binding Partners and Isolating Peptide Expressing Phage

In order to identify a phage expressing a peptide encoded by an exon, the library is screened with a binding partner. After identification of the phage displaying the binding partner-reactive peptide, the phage is isolated.

To facilitate the identification and isolation of the binding partner-bound peptide, the peptide or the binding partner (e.g., phage-displayed antibody) can be engineered as a fusion protein to include selection markers (e.g., epitope tags) or labels (defined above). Antibodies reactive with the selection tags (in the fusion proteins) or moieties that bind to the labels can then be used to isolate a peptide/binding partner complex via the eptiope or label. For example, a selection eptiope can be incorporated into the antibodies of an antibody display library that is used as a binding partner library to select expressed sequences. The peptide diplay library is incubated with the antibody display library to allow formation of peptide-displaying phage/antibody-displaying phage complexes. These complexes can be separated from non-reactive epitope-displaying phage using an antibody to the epitope tag. Similarly, a tag can be included in a fusion protein with a peptide in the peptide display library. Following incubation of the phage library with a binding partner and removal of unbound phage, an antibody (or other molecule that has affinity for the tag) can be used to isolate phage complexed with the binding partner.

A tag can also be used in an enrichment procedure, for example, to increase the proportion of open reading frames in a peptide display library. A library of phage comprising subsequences of genomic DNA will typically include a mixture of phage displaying peptides (in which the genomic subsequences cloned into the displaying peptides are in an open reading frame) and phage that do not display peptides (the cloned subsequences have an in-frame stop codon). In this enrichment procedure, a tag, e.g., an epitope tag, may be included in a phage display vector positioned such that the epitope tag is displayed only when there is an open reading frame in the cloned subsequence. The library generated from such a vector can then be enriched for potential exon-encoding subsequences by selecting phage that display the epitope tag using an antibody to the tag. The non-displaying phage are thus removed from the library population.

Detection and purification facilitating domains include, e.g., metal chelating peptides such as polyhistidine tracts and histidine-tryptophan modules that allow purification on immobilized metals, protein A domains that allow purification on immobilized immunoglobulin, or the domain utilized in the FLAGS extension/affinity purification system (Immunex Corp, Seattle Wash.). Any epitope with a corresponding high affinity antibody can be used, e.g., a myc tag (as used by e.g., Kieke (1997) Protein Eng. 10:1303-1310). See also Maier (1998) Anal. Biochem. 259:68-73; Muller (1998) Anal. Biochem. 259:54-61. The inclusion of a cleavable linker sequences such as Factor Xa or enterokinase (Invitrogen, San Diego Calif.) between the purification domain and binding site may be useful to facilitate purification. For example, an expression vector of the invention includes a polypeptide-encoding nucleic acid sequence linked to six histidine residues. One of the most widely used tags is six consecutive histidine residues or 6His tag. These residues bind with high affinity to metal ions immobilized on chelating resins even in the presence of denaturing agents and can be mildly eluted with imidazole. Another exemplary epitope tag is the E-tag (Pharmacia), used in Example 1, below. Selection tags can also make the epitope or binding partner (e.g., antibody) detectable or easily isolated by incorporation of, e.g., predetermined polypeptide epitopes recognized by a secondary reporter/binding molecule, e.g., leucine zipper pair sequences; binding sites for secondary antibodies; transcriptional activator polypeptides; and other selection tag binding compositions. See also Williams (1995) Biochemistry 34:1787-1797.

Screening by Multiple, Increasingly Stringent Rounds of Affinity Selection

Different “trapping” or approaches of increasing complexity, i.e., increasingly stringency, can be used to select binding partners capable of increasingly greater binding affinities. For example, these approaches can include use of multiple rounds of selection using monoclonal antibodies and/or polyclonal immune sera, followed by use of antibody phage-display libraries.

Use of decreasing concentrations of binding partner, e.g., antibody to “trap” peptide-displaying phage also selects for increased binding partner binding site affinity. As in Example 1, below, initial screens to trap 5q31 exon-displaying phage in the epitope library used commercially available monoclonal antibodies against an epitope known be encoded by the selected genomic fragment expressed by the epitope phage display library.

A variety of other parameters can be adjusted to select for high affinity binding sites, e.g., increasing salt concentration, temperature, and the like, can be used in combination with varying the type, quality and quantity of antibody binding reagents.

Antibody/peptide-displaying phage complexes can be separated from non-complexed peptide-displaying phage using antibodies specific for the antibody selection “tag,” e.g., E-tag (Pharamacia). The selected phages are then used to infect bacteria under selection pressure, e.g., antibiotics, selecting against generation of antibody-displaying phage. Thus, after antibiotic selection, only the epitope-displaying phage survive.

Such multiple rounds of selection “enriches” the library for the exon-containing clones. If 1% of the genome is coding, then a library with 10⁶ genomic insert-containing phage should contain about 10⁴ exon-containing clones. However, a given exon will only be correctly displayed in one out of six reading frames. Thus, approximately 500 clones of 10⁶ will express exons as polypeptides. If size selection (e.g., >150 bp) eliminates 90% of the intron sequences due to premature stop codons, then a library with 10⁶ insert-containing phage should be enriched by one to two orders of magnitude to contain approximately 5×10⁴ epitope-displaying clones. Analysis of phage display selections indicates that about one in 20 million epitope-displaying phage is capable of selectively reacting to an epitope-specific antibody after 3 to 4 rounds of selection. Thus, a final enrichment of exon:intron sequences greater than 1000:1 is anticipated after multiple rounds of selection. This enriched phage population will contain multiple copies of the same exon clone and clones of varying lengths. Variations in length can be used to fingerprint clone polymorphisms and to limit clones for further analysis.

Enriching can also be performed by making use of a phage library that expresses sequences that are from non-protein coding regions of the genome to select binding partners, e.g., antibodies, that are used to remove phage encoding such sequences from a library comprising both exon and non-coding subsequences of a genomic fragment. For example, a phage display library that expresses repetitive DNA sequences, e.g., Alu sequences or Kpn sequences, can be used to identify antibodies that recognize peptides encoded by the repetitive sequences, which peptides are normally not expressed in vivo. These antibodies can in turn be used to enrich a genomic phage display library comprising both coding and non-coding subsequences from a genomic fragment. Phage expressing the repetitive sequences will express peptides that bind to the enrichment antibodies, which are used to remove the phage from the library. Accordingly, the peptide phage display library is enriched for exon subsequences, i.e., sequences that encode protein in vivo.

Co-Selection of High Affinity Epitope-Binding Antibodies

Identification of epitopes using the methods of the invention also allows for rapid co-selection of high affinity epitope-binding antibodies. These epitope-specific antibodies are powerful reagents for functional genomic analyses. Additionally, the coupling of epitope trapping with rapid identification of epitope-binding antibody reagents facilitates high throughput identification of exons within a genomic region. These antibodies can also be used for immunohistochemistry, flow cytometric analyses, ELISAs, western blots, protein quantification and the like.

Isolating the Phage Nucleic Acid Insert

After identifying a phage expressing a protein epitope specifically reactive with the selected binding partner, the insert encoding the protein epitope is isolated. The trapped epitope-expressing phage can contain as inserts either exonic genomic nucleic acid or cDNA sequence encoding epitope coding region. Inserts can be isolated by restriction digest of isolated phage nucleic acid, amplification (e.g., PCR), or other well known methods, as described below. Inserts can be further amplified and/or subcloned for mapping purposes, as discussed below.

Mapping Genomic Sequences

Genomic mapping is the identification of the physical location of a nucleic acid sequence on a specific chromosome. Mapping can determine the physical relationship of a gene to a genetic linkage map or other relevant chromosomal landmarks, such as banding patterns or chromosomal rearrangements. In the methods of the invention, the sequence of the insert of a phage that displays a peptide bound by a binding partner is typically determined. The sequence information can be used to identify the specific region of the chromosome that harbors the exon. In applications in which the sequence of the chromosomal region is already available, the position of the exon in the genomic fragment can readily be determined. The sequence of that regions can then further be analyzed, e.g., to detect the gene that comprises the exon.

Sequencing of Nucleic Acid

Sequencing of newly isolated genomic DNA will identify and characterize epitope-encoding nucleic acid. Sequencing of isolated epitope-encoding nucleic acid will also identify possible functional characteristics of the sequences, such as, e.g., coding sequences for oncogene polypeptides, trans-acting transcriptional regulators, and the like.

Nucleic acid sequences can be sequenced as inserts in vectors, as inserts released and isolated from the vectors or in any of a variety of other forms (i.e., as amplification products). Inserts can be released from the vectors by restriction enzymes or amplified by PCR or transcribed by a polymerase. For sequencing of the inserts, primers based on the N- or C-terminus, or based on insertion points in the original phage or other vector, can be used. Additional primers can be synthesized to provide overlapping sequences. A variety of nucleic acid sequencing techniques are well known and described in the scientific and patent literature, e.g., see Rosenthal (1987) supra; Arlinghaus (1997) Anal. Chem. 69:3747-3753, for use of biosensor chips for sequencing; Pastinen (1996) Clin. Chem. 42:1391-1397; Nyren (1993) Anal Biochem. 208:171-175.

Additional Physical Mapping Techniques

The sequence can also be mapped using additional techniques. Typically, physical mapping strategies organize individual genomic fragments, such as the exon-encoding genomic sequences identified by the methods of the invention, into a high-resolution map of continuous overlapping fragments, or “contigs.” A variety of methodologies for mapping genomic sequences are well known in the scientific and patent literature. Examples include fingerprinting inserts by electrophoretic sizing of restriction fragments (Stallings (1991) Genomics 10:807-815); or hybridizing genomic fragments or oligonucleotides to overlapping, known and mapped genomic clones fixed to filters or arrays (see, e.g., Craig (1990) Nucleic Acids Res. 18:2653-2660; Shalon (1996) supra; Sapolsky (1996) Genomics 33:445-456; Ramsay (1998) Nat. Biotechnol. 16:40-44; Boehm (1998) Methods 14:152-158.

Nucleic Acid Hybridization Techniques

Hybridization techniques can be used in the methods of the invention, e.g., to map identified and isolated epitope-encoding genomic sequences, as on arrays or filters, to additionally confirm or analyze mRNA message, and the like. A variety of methods for specific DNA and RNA measurement using nucleic acid hybridization techniques are known to those of skill in the art. See, e.g., Nucleic Acid Hybridization, A Practical Approach, Ed. Hames, B. D. and Higgins, S. J., IRL Press, 1985; Sambrook, Tijseen. One method for evaluating the presence or absence of specific nucleic acid sequence, e.g., an antibody- or epitope-encoding nucleic acid, in a sample involves a Southern transfer. In a Southern Blot, a genomic or cDNA (typically fragmented and separated on an electrophoretic gel) can be hybridized to a probe specific for the target region. Comparison of the intensity of the hybridization signal from the probe for the target region with the signal from a probe directed to a control region provides an estimate of the relative copy number of the target nucleic acid. cDNA generated from RNA message by reverse transcription and amplification can also be measured in this manner. Similarly, a Northern transfer can be used for the detection of RNA message. Typically, RNA is isolated from a given cell sample using an acid guanidinium-phenol-chloroform extraction method. The RNA is electrophoresed to separate different species and transferred from the gel to a nitrocellulose membrane, where it is probed by hybridization or PCR.

Sandwich assays are commercially useful hybridization assays for detecting or isolating protein or nucleic acid. Such assays utilize a “capture” nucleic acid or protein that is often covalently immobilized to a solid support and a labeled “signal” nucleic acid, typically in solution. A clinical or other sample provides the target nucleic acid or protein. The “capture” nucleic acid or protein and “signal” nucleic acid or protein hybridize with or bind to the target nucleic acid or protein to form a “sandwich” hybridization complex. To be effective, the signal nucleic acid or protein cannot hybridize or bind substantially with the capture nucleic acid or protein.

Typically, nucleic acids are labeled with a detectable composition to detect hybridization. Complementary probe nucleic acids or signal nucleic acids may be labeled and detected by any method. Useful labels include, e.g., ³²P, ³⁵S, ³H, ¹⁴C, ¹²⁵I, ¹³¹I; fluorescent dyes (e.g., FITC, rhodamine, lanthanide phosphors, Texas red), electron-dense reagents (e.g. gold), enzymes, e.g., as commonly used in an ELISA (e.g., horseradish peroxidase, beta-galactosidase, luciferase, alkaline phosphatase), colorimetric labels (e.g. colloidal gold), magnetic labels (e.g. Dynabeads™), biotin, dioxigenin, or haptens and proteins for which antisera or monoclonal antibodies are available. The label can be directly incorporated into the nucleic acid, peptide or other target compound to be detected. Alternatively, it can be attached to a probe or antibody which hybridizes or binds to the target, such as a “selection tag” of a recombinant, phage-displayed antibody binding site molecule, as discussed below.

The detection can be by, e.g., spectroscopic, photochemical, biochemical, immunochemical, physical or chemical means. Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal, i.e., antibody-antigen or complementary nucleic acid binding. The label may also allow indirect detection of the hybridization complex. For example, where the label is a hapten or antigen, the sample can be detected by using antibodies. In these systems, a signal is generated by attaching fluorescent or radioactive label or enzymatic molecule to the antibodies. The sensitivity of the hybridization assays can be enhanced through use of a target nucleic acid or signal amplification system which multiplies the target nucleic acid or signal being detected. Alternatively, sequences can be generally amplified using nonspecific PCR primers and the amplified target region later probed for a specific sequence indicative of a mutation.

In situ Hybridization

An alternative means for mapping of a peptide-encoding sequence or evaluating the level of expression of a peptide-encoding sequence is in situ hybridization. In situ hybridization assays are well known (e.g., Angerer (1987) Methods Enzymol 152:649). Generally, in situ hybridization involves fixation of tissue or biological structure to analyzed; prehybridization treatment of the biological structure to increase accessibility of target DNA, and to reduce nonspecific binding; hybridization of the mixture of nucleic acids to the nucleic acid in the biological structure or tissue; posthybridization washes to remove nucleic acid fragments not bound in the hybridization; and, detection of the hybridized nucleic acid fragments. The reagent(s) used in each of these steps and their conditions for use vary depending on the particular application. In a typical in situ hybridization assay, cells are fixed to a solid support, as a glass slide. The cells can be denatured with heat or alkali. The cells are then contacted with a hybridization solution at a moderate temperature to permit annealing of labeled probes specific to the nucleic acid sequence. The probes can be labeled, e.g., with radioisotopes, fluorescent reporters and the like. Hybridization capacity of repetitive sequences can be also blocked. Hybridization protocols are described, e.g., in Pinkel (1988) Proc. Natl. Acad. Sci. USA 85:9138-9142; Methods in Molecular Biology, Vol. 33: In Situ Hybridization Protocols, Choo, ed., Humana Press, Totowa, N.J. (1994); Kallioniemi (1992) Proc. Natl Acad Sci USA 89:5321-5325; Zhang (1994) Science 277:383.

Another well-known in situ hybridization technique is the so-called “FISH” or “fluorescence in situ hybridization,” well known in the art, described by, e.g., Macechko (1997) J. Histochem. Cytochem. 45:359-363; Raap (1995) Hum. Mol. Genet. 4:529-534. Hybridization of chromosomes typically uses dual color FISH, in which two probes are utilized, each labeled by a different fluorescent dye. A test probe that hybridizes to the region of interest is labeled with one dye, and a control probe that hybridizes to a different region (e.g., a centromere) is labeled with a second dye. A nucleic acid that hybridizes to a stable portion of the chromosome of interest, or another chromosome, is often most useful as the control probe. In this way, differences between efficiency of hybridization from sample to sample can be accounted for. FISH methods for detecting chromosomal abnormalities can be performed on nanogram quantities of the subject nucleic acids. One variation of FISH, using digital imaging microscopy, can identify a single RNA molecule, see Femino (1998) Science 280:585-590.

Nucleic Acid Arrays

Nucleic acid hybridization assays for the detection and mapping of peptide-encoding sequences, for quantitating copy number, for sequencing, and the like, can also be performed in an array-based format. Arrays are a multiplicity of different “probe” or “target” nucleic acids hybridized with a sample nucleic acid. For example, the fixed probe can be a physically mapped genomic sequence and the sample nucleic acid can be an epitope-encoding genomic insert from a phage isolated by the methods of the invention. In an array format a large number of different hybridization reactions can be run essentially “in parallel.” This provides rapid, essentially simultaneous, evaluation of a wide number of samples. A genomic fragment encoding an epitope can be hybridized to an array comprising thousands of defined, physically mapped genomic fragments. For example, the genomic sequence of the budding yeast Saccharomyces cerevisiae has been used to synthesize high-density oligonucleotide arrays for monitoring the expression levels of nearly all yeast genes. This parallel approach involves the hybridization of total mRNA to a set of arrays that contain a total of more than 260,000 specifically chosen oligonucleotides synthesized in situ using light-directed combinatorial chemistry (Wodicka (1997) Nat. Biotechnol. 15:1359-1367). Methods of performing hybridization reactions in array based formats are well known to those of skill in the art, see, e.g., Pastinen (1997) Genome Res. 7:606-614; Shalon (1996) Genome Res. 6:639-645; Jackson (1996) Nature Biotechnology 14:1685; Chee (1995) Science 274:610; WO 96/17958.

Phage Expression Vectors

The invention also provides a novel phage expression vector for constructing display libraries. The vector comprises a polylinker region, an out-of-frame pIII gene, at least one non-palindromic rare cutting restriction enzyme site located in the polylinker site, and an epitope tag. The non-palindromic rare cutting restriction enzyme site should only be located within the polylinker site (no such sites outside the polylinker region). In one embodiment, the non-palindromic rare cutting restriction enzyme site is an SfiI site. This novel vector addresses the critical factors needed in construction of useful and quality phage expression vector libraries. They include, e.g., minimal vector background, successful bacterial transformation and display of unique marker tags.

To further attenuate the contribution of background vector it is also desirable to engineer a phage expression vector that cannot express its own coat protein. During epitope library construction, any vector religation without insert will decrease the diversity of the library. Thus, the ability of the phage expression vector to prevent such religation is a critical component. The vector of the invention, by providing a non-palindromic rare cutting restriction enzyme site located in the polylinker site, solves this problem. The in-frame pIII coat protein gene was frame-shifted to become “out of frame,” thus generating a non-coat protein-displaying phage. The non-palindromic cloning site prevents sticky-end religation and decreases the requirement for vector phosphorylation, which often reduces transformation efficiency. In one embodiment, the phage expression vector of the invention includes two SfiI sites, a polylinker site and an out-of-frame pIII gene, wherein the SfiI sites are located in the polylinker site.

The vector of the invention also contains a selection tag encoding sequence, where the tag aids in the identification and/or the isolation of the phage of interest. The tag can be, e.g., an epitope tag or an antibiotic resistance gene. The epitope tag can be, e.g., a metal chelating peptide tag (e.g., polyhistidine tag), a myc tag, or a protein A domain, as described above. The selection tag can also be a gene encoding an antibiotic resistance polypeptide, such as ampicillin, chloramphenicol, kanamycin, bleomycin, or hygromycin.

In one embodiment, the M13 phage vector pHEN-1 (Hoogenboom (1991) Nuc. Acids Res. 19:4133-4137) is used as the backbone for the construction of the vector of the invention. The leader, polylinker and antibiotic resistance sequences of pHEN-1 are redesigned. The resultant novel vector of the invention is designated pBPM-1.

Construction of an SfiI cloning site in pHEN-1 requires removal of its SfiI site from the leader sequence. To further attenuate the contribution of background vector, pHEN-1's in-frame pIII gene is frame-shifted to become an out of frame and thus non-displaying phage. Two new markers are added to facilitate identification and isolation of the epitope-displaying phage. The first is a 5′ polyhistidine tag, e.g., a hexahistidine (His₆) sequence, to act as a second epitope marker for displayed peptides. A second antibiotic marker, chloramphenicol resistance gene, is added to allow selection and differentiation of epitope from antibody libraries.

In summary, the phage expression vector of the invention based on pHEN-1 or an analogous phage expression vector includes: a substitutional mutation to destroy the SfiI site in the leader sequence; excision of the NcoI-NotI polylinker; replacement of polylinker region with a new NcoI-NotI oligo polylinker which contains a 5′ hexahistidine epitope tag, the addition of two SfiI cloning sites and single distal 3′ base deletion, and insertion of a chloramphenicol acetyltransferase gene adjacent to the Amp region. Thus the final vector will allow for display of SfiI-SfiI inserts with a N-terminal His tag and a C-terminal myc tag with antibiotic selectivity.

Libraries Expressing Normally Non-Transcribed Genomic Sequences

The invention also provides a phage library displaying protein epitopes encoded by genomic nucleic acid sequences which do not normally generate polypeptides in vivo. These libraries can be used to produce antibody phage display libraries displaying antibodies specifically reactive with such “junk” protein.

The majority of chromosomal nucleic acid is not protein-encoding sequence. For example, in mammals, the vast majority of intronic sequences are not normally transcribed. However, fragments of intronic sequences, when inserted in expression vectors operationally linked to transcriptional regulatory elements, can be transcribed and translated to protein. Genomic nucleic acid sequences such as repetitive sequences, e.g., LINES and SINES, such as Alu repeat sequences or Kpn repeat sequences (Sun (1984) Nucleic Acids Res. 12:2669-2690), which are not normally transcribed, can be similarly cloned and induced to expressed such “junk” protein. The Alu repeat sequence alone is estimated to account for 5% of human genomic DNA, see, e.g., Yulug (1997) Genomics 27:544-548. Thus, expression of randomly fragmented genomic nucleic acid as inserts in expression vectors will generate significant amounts of protein not representative of polypeptides expressed in vivo.

Frequently, as in the methods of the invention, an objective is to select phages displaying naturally expressed peptides capable of specifically reacting with a binding partners. When the epitope phage display libraries are generated using randomly fragmented genomic DNA, phages expressing such “junk” protein will be produced. These phages will produce undesirable background when trying to identify phage-displayed epitopes capable of specifically interacting with the binding partner. Thus, elimination of the junk protein-displaying phages before the epitope-binding site screening step can be a helpful in reducing such unwanted background. Libraries of antibodies reactive with such junk protein can be used to pre-screen epitope phage display libraries before their screening for reactivity with binding sites. The invention provides for such libraries in the form of antibody phage display libraries. The invention also provides epitope phage libraries displaying such junk protein to generate and select for these corresponding antibody libraries.

Non-transcribed genomic sequences can be generated using any variety of recombinant or synthetic methods, as described above. See also Hwu (1986) Proc. Natl. Acad. Sci. USA 83:3875-3879; Britten (1988) Proc. Natl. Acad. Sci. USA 85:4770-4774; Shen(1991) J. Mol. Evol. 33:311-320.

### EXAMPLES

### Example 1

A phage display library comprising subsequences of genomic fragments from a 50 kb human P1 artificial chromosome, which contains genes from the 5q31 Interleukin gene cluster, was used to demonstrate that protein-encoding regions of the genomic fragment can be identified.

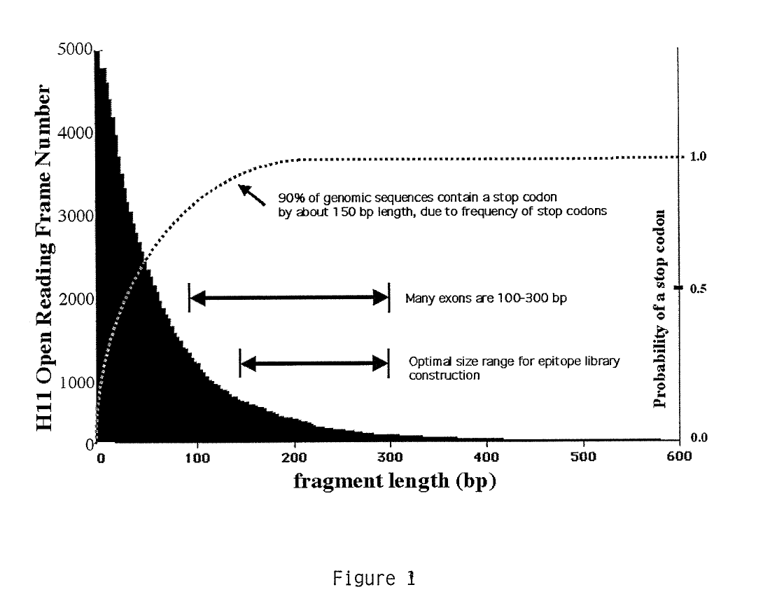

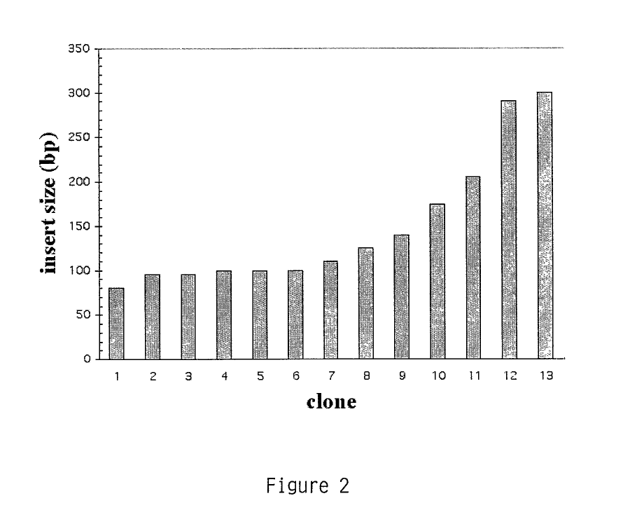

An epitope phage display library, optimized to contain exon-sized inserts, was generated from a 50 kb P1/BAC clone that contained human Interleukin-4, Interleukin-13, and kinesin-like protein-3. The genomic DNA was randomly fragmented using DNAse I and fragments approximating 100-300 bp were isolated by gel electrophoresis and cloned into the pORF-1 vector, which contains a 5′ hexahistidine tag, an asymmetric Sfi-1 cloning site, a 3′ amber codon and C-terminal c-myc epitope tag. The fragment sizes were selected to maximize enrichment of exons (FIG. 1). Selection of the target insert size range to maximize exon display was based upon in silico analyses of the size distribution of exons in genes within the H11 P1 (FIG. 1). Long fragments (>300 bp) are more likely to contain intron sequence with stop codons, which would prevent translation of displayed protein (FIG. 1), thereby reducing the diversity and complexity of the library. However, short fragments have a lower likelihood of folding into a domain structure, which could mimic the conformational epitopes that antibodies typically recognize. Thus, while longer fragments are better for domain structure (domain size typically 80-110 amino acids), the potential problems with introns and stop codons suggests that 100-300 bp is optimal. The size distribution of fifteen random, unselected clones was determined using PCR. The majority of clones (12/15) contained an average insert size of 150 bp with a range of 80-300 bp (FIG. 2). DNA sequencing of random clones revealed fragments of genomic sequence in both coding orientations. Approximately 5/13 random clones contained DNA sequence that corresponded to E. coli genomic sequence and 8/13 clones contain human intron genomic sequence. Vector religation occurred in 20% (3/15) of clones. The library of 2×l0⁶ clones appeared to be sufficiently large to cover the sequence space anticipated for a 50-100 kb BAC library (<<10⁵ clones) and contained fragment sizes in the desired exon-size range.

Antibody Selection of H11 Genomic Library Members

Enrichment of exon-based epitope sequences, corresponding to genes within the b 5q31/H11 locus, was demonstrated by selecting the genomic epitope library using antibodies specific for the proteins encoded by 5q31/H1 exons. Monoclonal (Mab604) and polyclonal (C19) antibodies against Interleukin-4 were used for epitope selection. The C19 antibody was raised against the C-terminal peptide of IL-4 and corresponds to exon 4 of IL-4. Significant enrichment of the H11 library occurred after two rounds of selection against all three antibodies, as indicated by increasing phage titers (1-3 orders of magnitude per selection round). More than 50% of individual clones screened by phage-ELISA were positive after the second round of selection.

DNA sequencing revealed unique clones against each antibody. Most clones contained similar sized inserts. The DNA sequence of fifteen positive clones was determined. Two unique clones were identified using C19 anti-IL-4 antibody selection. One clone (H11_{—}207) matches the human Interleukin-4 epitope consisting of an IL-4 fusion product composed of a 46 bp human telomeric sequence (2PTEL066, 176-130 bp) and the IL4 cDNA sequence from exon 4 (AC004039, 24244-24170 bp). Another clone insert corresponded to E. coli genomic DNA (e.g., clone H11_{—}201). The Mab604 anti-IL4 antibody selection resulted in isolation of two unique clones of 800 bp corresponding to a contaminating human single-chain antibody sequence.

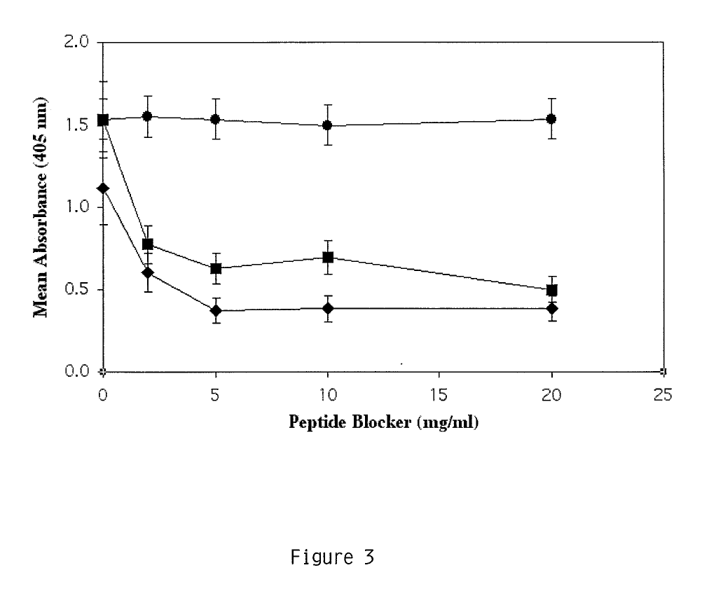

The specificity of phage clones for the human IL-4 epitope was demonstrated by competition ELISA using the specific C19 blocking peptide, SC-1260. Binding of both the IL-4 epitope (clone H11_{—}207) and the IL-4 mimotope (clone H11_{—}201) to antibody was displaced with increasing concentrations of peptide, confirming the IL-4 specificity of the phage epitopes (FIG. 3).

Genomic Epitope Library Construction and Characterization

The H11 library described above was constructed from a 50 kb human P1 (P1 clone 876h9, Genbank accession AC004039), containing the Interleukin-4, Interleukin-13, and kinesin-like protein-3 genes from 5q31. 20 μg P1 DNA was purified by standard method (Qiagen) (Collins et al., Proc. Natl. Acad. Sci USA 95:8703-8708, 1998) and was randomly fragmented with decreasing concentrations of DNAse I (10 units/ml) in 10 mM Tris pH 7.0/10 mM MnCl₂ for 8 minutes at 15° C., extracted and precipitated. Fragments were blunted with 5 units/μg T4 polymerase for 30 min at 12° C., extracted and precipitated. Linkers containing a Sfi-1 restriction site (Link1 5′-AGCGGCCGCAGGCCATGGAGGCC-3′, Link2 5′-GGCCTCCATGGCCTGCGGCCGCT-3′) were ligated to target DNA with 400 units T4 DNA ligase for 2 hours at room temperature. The resulting product was electrophoresed on a 2.0% agarose gel and the size range of 100-300 bp was collected and eluted from NA-45 DEAE paper (Schleicher and Schuell, Keene, N.H.) 100 ng of the linker-ligated product was used as template in PCR with a nested primer LP5 (5′-GCGGCCGCAGGCCATGGA-3′) with 2.5 units Pfu Polymerase/2.5 units panoTAQ for 30 cycles (94° C.×1 min, 55° C.×1 min, 72° C.×1 min). The PCR products were digested with Sfi-1 and gel purified. A positive control phage displaying the 3′ exon of the IL-4 cDNA (490-612 bp) was also constructed (Yokota et al., Proc. Natl. Acad. Sci USA 83:5894-5898, 1986).

A phage display vector, pORF-1, was engineered for gene fragment phage display. It is a pHEN-1 (Hoogenboom et al., Nucl. Acid Res. 19:4133-4137, 1991) based vector that contains a pelB leader sequence, a 5′ hexahistidine tag and a non-religatable Sfi-1 insert cloning site which is upstream and contiguous with the M13 gene III and a 3′ myc epitope tag. pORF-1 was constructed by two rounds of template mutagenesis of pHEN-1 vector with primers (NSFI 5′-GCGGCCCAGCCGGCGATGGCCCAGCACCATCACCATC ATCACGGGGCCATGGTGCAGCTGCAGG-3′; SUP 5′-TCACGGGGCCATGGG GGCCCAGGCCTCAGTCGATCGACACGGCCTCCACGGCCGCAGAACAA-3′) (Kunkel et al. J. Biol. Chem 263:14784-14789, 1988). The base vector contained an out-of-frame 1 kb stuffer fragment. Sfi-1 digested insert was ligated into the digested vector and optimized ligation products were electroporated into E. coli TG-1. The size distribution of library inserts was evaluated by PCR with primers flanking the cloning site (Sfiseq5, 5′-TCACCATCATCACGGGGCCAT-3′ and Sfiseq3, 5′-GTTTTTGTTCTGC GGCCGTTG-3′) with Pfu Polymerase for 30 cycles (94° C.×1 min, 55° C.×1 min, 72° C.×1 min).

Selection and Screening of H11 Epitope Library

Antibodies specific for human IL-4 (C19; Santa Cruz Biotechnology, Santa Cruz, Calif.) (Mab604; R&D Systems, Minneapolis, Minn.), and IL-13 (IL13C; Santa Cruz Biotechnology) were purchased from commercial sources. Epitope selections were performed as previously described (Mullaney and Pallavicine, 2001, supra; Schier et al., J. Mol. Biol. 263:551-567, 1996) using (50 μg/ml) antibody-coated immunotubes (Nunc). Random clones from the second round of selection were screened by phage-ELISA on microtiter plates (Corning) coated overnight at 4° C. with 25 μg/ml of antibody. Binding of phage was detected with 1:1000 horseradish peroxidase-conjugated anti-M13 (Amersham Pharmacia, Piscataway, N.J.). Phage displaying epitopes did not cross-react with plastic, albumin, or IgG as determined by ELISA. Positive controls included an IL-4 phage. Insert size of ELISA positive clones was determined by PCR and clones with unique insert size were DNA sequenced and aligned by BLAST. Selections were repeated in cases where no enrichment occurred.

Determination of Epitope Clone Specificity

The specificity of phage epitope clones for the human IL-4 epitope was determined by competition ELISA using a specific blocking peptide, SC-1260 (Santa Cruz Biotechnology), corresponding to the epitope for the anti-IL-4 antibody C19. ELISA was performed as described above, except that the C19 antibody was preincubated with increasing concentrations (0 to 20 mg/ml) of SC-1260 prior to incubation with phage epitopes. A phage displaying coverage of the 3′ exon of the IL-4 cDNA served as positive control.

Summary

The advantages of the methods of the invention were demonstrated by epitope “trapping” genomic sequence from the 5q31 region of human chromosome 5 using monoclonal, polyclonal and antibody phage display libraries specific for proteins expressed in hemopoietic cells. It was thus demonstrated that the methods of invention can rapidly identify, isolate and map genes encoding polypeptides expressed by these hematopoietic cells. As a specific example, an exon-encoding genomic fragment encoding interleukin-4 (IL-4) was isolated and mapped.

An epitope phage display library expressing 5q31 sequences was chosen because 5q31 is a chromosomal region known to contain clusters of cytokine gene families. They include e.g., interleukin 3 (IL-3), IL-4, IL-5, IL-9, IL-13), granulocyte macrophage colony stimulating factor (GM-CSF), novel putative transcription factors, metabolic proteins and cell cycle related proteins (Frazer (1997) Genome Res. 7:495-512). This epitope phage display library was screened with an antibody phage display library generated by immunizing mice with hemopoietic cells. Identification of genomic DNA encoding proteins expressed by the hemopoietic cells used in the immunization, e.g., IL-4 and IL-13, is demonstrated. These studies on 5q31 establish that the methods of the invention, using epitope trapping, are a rapid and efficient method to identify genes expressing polypeptides in specific cells or target tissues.

Production of antibody phage which produce high affinity anti-IL-4 and IL-13 scFVs also confirms the utility of “epitope trapping” methods of the invention to generate antibody tools for functional analyses.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of identifying an exon in a eukaryotic genomic fragment, the method comprising:
expressing a population of subsequences of the genomic fragment in a phage display library, wherein the population comprises protein-encoding subsequences and noncoding subsequences;
screening the phage display library with a binding partner to identify an expressed subsequence that specifically binds to the binding partner; and
mapping the expressed subsequence to the physical location in the genomic fragment, thereby identifying the exon.

2. The method of claim 1, wherein the binding partner is an antibody, an enzyme or a receptor.

3. The method of claim 2, wherein the binding partner is an antibody.

4. The method of claim 3, wherein the antibody is a single chain antibody.

5. The method of claim 1, wherein the binding partner is expressed by a phage display library.

6. The method of claim 5, wherein the phage display library is an antibody phage display library generated using mRNA isolated from a stimulated B cell or a naïve B cell.

7. The method of claim 6, wherein mRNA isolated from the stimulated B cell is MRNA isolated from a stimulated splenic B cell that is isolated from an animal immunized with a composition comprising the protein epitope encoded by the genomic sequence or a nucleic acid encoding the protein epitope.

8. The method of claim 1, wherein the expressed subsequences are from about 100 base pairs to about 300 base pairs in length.

9. The method of claim 1, wherein the genomic fragment is from a mammalian genome.

10. The method of claim 1, further wherein the exon is abnormally expressed in a cell of an individual with a disease or condition.

11. The method of claim 10, wherein the cell has a genomic translocation involving the exon sequence.

12. The method of claim 10, wherein the disease is cancer.

13. The method of claim 1, further comprising a step of enriching for phage expressing subsequences of the genomic fragment that are exons.

14. The method of claim 13, wherein the step of enriching comprises incubating the phage library with a binding partner specific for a peptide encoded by a subsequence that does not encode a peptide in vivo, and removing phage expressing the peptide from the library.

15. The method of claim 14, wherein the subsequence that does not encode a peptide in vivo is a repetitive sequence.

16. The method of claim 15, wherein the repetitive sequence is an Alu sequence or a Kpn sequence.

17. A phage display library comprising phage that express a population of subsequences of a eukaryotic genomic fragment, wherein the population comprises protein coding subsequences and noncoding subsequences.

18. The phage display library of claim 11, wherein the eukaryotic genomic fragment is from a mammalian genome.

19. The phage display library of claim 17, wherein the library is constructed using a pBPM-1 vector.

20. The phage display library of claim 17, wherein the expressed subsequences are from about 100 base pairs to about 300 base pairs in length.

21. A phage expression vector comprising a polylinker region, an out-of-frame pIII gene, and at least one non-pallindromic rare cutting restriction enzyme site located in the polylinker site, wherein the non-pallindromic rare cutting restriction enzyme site is not located outside the polylinker region, and a selection tag encoding sequence.

22. The phage expression vector of claim 21, wherein the non-pallindromic rare cutting restriction enzyme site is an SfiI site.

23. The phage expression vector of claim 21, wherein the selection tag is an epitope tag selected from the group consisting of a polyhistidine tag or a myc tag.

24. The phage expression vector of claim 21, wherein the selection tag is an antibiotic resistance polypeptide.

25. A method of identifying an exon in a genomic fragment, the method comprising:
expressing a population of subsequences of the genomic fragment in a phage display library, wherein the population comprises protein-encoding subsequences and noncoding subsequences;
enriching for phage expressing subsequences of the genomic fragment that are exons;
screening the phage display library with a binding partner to identify an expressed subsequence that specifically binds to the binding partner; and
mapping the expressed subsequence to the physical location in the genomic fragment, thereby identifying the exon.

26. The method of claim 25, wherein the step of enriching comprises incubating the phage library with a binding partner specific for a peptide encoded by a subsequence that does not encode a peptide in vivo, and removing phage expressing the peptide from the library.

27. The method of claim 26, wherein the subsequence that does not encode a peptide in vivo is a repetitive sequence.

28. The method of claim 25, wherein the expressed subsequences are from about 100 base pairs to about 300 base pairs in length.
